(19) United States
(12) Patent Application Publication
(10) Pub. No.: US 2025/0100202 A1
(43) Pub. Date: Mar. 27, 2025

(54) EXTRUDER FOR HYBRID MICROCATHETER

(71) Applicant: BALT EXTRUSION, Montmorency (FR)

(72) Inventor: Léopold PLOWIECKI, Montmorency (FR)

(21) Appl. No.: 18/975,104

(22) Filed: Dec. 10, 2024

Related U.S. Application Data
(63) Continuation of application No. PCT/EP2023/065486, filed on Jun. 9, 2023.

(30) Foreign Application Priority Data
Jun. 10, 2022 (EP) 22305849.

(51) Int. Cl. B29C 48/49; A61M 25/00; B29C 48/09; B29C 48/19; B29C 48/50; B29L 31/00

(52) U.S. Cl. CPC B29C 48/49; A61M 25/0009; B29C 48/09; B29C 48/19; B29C 48/501; A61M 2025/0042; B29L 2031/7542

(57) ABSTRACT

An extruder for hybrid microcatheters comprises an extrusion die, a commuter element and several resin sources, the commuter clement allowing for resin selection.

### CROSS-REFERENCE TO RELATED APPLICATIONS

This application is a continuation of International Patent Application PCT/EP2023/065486, filed Jun. 9, 2023, which claims the benefit of foreign priority to European Patent Application 22305849.6, filed Jun. 10, 2022, with the entire contents of each of these applications hereby incorporated by reference herein.

### FIELD

The present disclosure relates to microcatheter fabrication, in particular to microcatheters having different flexibility along their length. This disclosure relates to extruders, process of fabrication of catheters and microcatheters. Such catheters are particularly adapted to the field of interventional radiology and neuroradiology.

### BACKGROUND

Microcatheter technology has advanced from the 1980's to the point that a microcatheters are now commonly used in the treatment of vascular lesions of the vascular central system. Microcatheters are low profile catheters used to treat strokes, cerebral aneurysms, fistulas, arterial venous malformations, and other areas by occluding the pathologic vascular abnormality through an endovascular approach utilizing selective deposition of coils, particles, balloons, or liquid adhesives but also by opening occluded vessels for ischemic stroke. Such microcatheters can also be used in other areas of the body.

In these delicate procedures, a major difficulty involves steering the catheter through the tortuous bifurcations of the arteries to navigate it to the site of treatment. The ability to steer the catheter precisely and quickly is essential for effective catheterizations with minimal risks.

A key feature of catheters to achieve correct steering is their flexibility. Indeed, catheter need to be rigid in some parts, so that catheter may be directed precisely when pushed on its guide. But catheters need also to be flexible in some areas, where brain vascularity is highly tortuous. This feature must be realized with catheters of very small diameter, below 6 Fr, so as to be able to navigate through carotid into brain vessels.

An approach to obtain such catheters uses welding technology: at least two tubes made of different materials are welded where flexibility is expected to change. However, welding usually leads to changes in internal diameter, resulting in unpredictable changes in functional properties of the catheter. Moreover, welding lines are mechanically weaker than bulk material and welded catheters may break.

Extrusion of tubes with different resin sources is also know, leading to catheters with a gradient of composition along their length. This gradient of composition may be selected to provide with a gradient of flexibility. However, this extrusion technology usually leads to very long transition lengths, much longer than required in the tortuous arteries of brain.

Although catheters with variable flexibility are known, there is still a need for microcatheters, whose flexibility varies on very short length, typically less than 30 mm.

### SUMMARY

This invention thus relates to an extruder for thermoplastic hybrid microcatheters. The extruder comprises an extrusion die comprising a die member and a mandrel which is disposed along the same direction as the die member. The extruder further comprises at least two resin sources and a commuter element abutting the extrusion die and comprising at least one inflow channel opening on the mandrel. In addition, the commuter element is sliding on the extrusion die to allow flow from one resin source selected from the at least two resin sources to the die member through an extrusion channel delimited by the die member, the mandrel and the opening of the inflow channel on the mandrel. Besides, the extrusion channel and the inflow channel together have a volume Vᵢₙ lower than 20 mm³, preferably lower than 15 mm³.

In a first configuration, the commuter element is rotationally sliding on the extrusion die around the extrusion die axis. In this configuration, the extruder may comprise three resin sources.

In a second configuration, the commuter element is longitudinally sliding on the extrusion die.

In an embodiment, the dimensions of the die member and mandrel are selected in the following couples (in mm): 0.4/0.15; 0.6/0.25; 0.8/0.4; 1/0.6; 1.2/0.8; 1.4/1; 1.6/1.2; 1.8/1.4; 2/1.6 2.2/1.8; 0.8×1.2/0.6; 0.6×1/0.4; 1×1.4/0.8; 1.2×1.6/1; 1.5×1.9/1.2; 1.8×2.2/1.5.

In an embodiment, the extrusion die comprises a die member and two mandrels. Preferably, the dimensions of the die member, first mandrel and second mandrel are selected in the following triplets (in mm): 0.6/0.15/0.15; 1/0.4/0.4; 1.4/0.6/0.4; 1.8/0.8/0.6; 2.2/1/0.8; 0.8×1.2/0.4/0.4; 1×1.4/0.6/0.4; 1.5×1.9/0.8/0.4.

In an embodiment, the mandrel has a central cavity that extends in the axial direction, said central cavity of the mandrel being connected to a gas source.

The invention also relates to a process of extrusion of thermoplastic hybrid microcatheters comprising the following steps:
placing the commuter element of an extruder as disclosed hereabove to allow flow from a first resin source to the die member;
extruding a predetermined length of microcatheter;
sliding the commuter element to allow flow from a second resin source to the die member; and
extruding a predetermined length of microcatheter.

In an embodiment, extrusion speed is comprised between 30 mm·s⁻¹ and 200 mm·s⁻¹.

Another object of the invention is a microcatheter obtained by the process disclosed hereabove, wherein the couple of resins used are selected from:
polypropylene and a mixture of polypropylene with thermoplastic polyurethane in a weight ratio comprised between 20:80 and 80:20;
polyamide and polyamide-polyester bloc copolymers; or
two different types of polyvinylchloride.

In an embodiment, transition length between two resins is lower than 25 mm, preferably lower than 20 mm.

### DEFINITIONS

In the present invention, the following terms have the following meanings:

“Dimensions”, in reference to a die member, a mandrel, a tube or a catheter, refers to the diameter if the element is cylindrical, or refers to the short and long axis if the element is ellipsoidal or oval shaped—noted short axis×long axis. Dimensions for a couple of one die member and one mandrel is noted as dimension of the die/dimension of the mandrel. For instance, if the die has an ellipsoidal shape with short axis of 0.8 mm and long axis of 1.2 mm noted 0.8×1.2; and the mandrel is cylindrical with a diameter of 0.6 mm, the couple will be noted 0.8×1.2/0.6. The same applies for a triplet comprising one die member and two mandrels. For instance, if the die has an ellipsoidal shape with short axis of 0.8 mm and long axis of 1.2 mm noted 0.8×1.2; and the two mandrel are cylindrical with a diameter of 0.4 mm, the triplet will be noted 0.8×1.2/0.4/0.4.

“French”, abbreviated Fr, refers to the catheter scale for diameters: 1 mm equals 3 Fr. Therefore, the diameter of a round catheter in millimeters can be computed by dividing the French size by 3.

“Hybrid”: refers to a microcatheter whose composition is not uniform. Some parts of the microcatheter are made of a first material A, and other parts of the microcatheter are made of a second material B. B is different from A in terms of flexibility. Part of microcatheter where composition changes from first material A to second material B is a transition. A hybrid microcatheter may comprise more than two different materials.

“Resin”: refers to the material used to prepare microcatheters, in pellet form or in molten from. By extension, resin also refers to the material of the microcatheter. Therefore, the terms resin and material are used interchangeably.

“Transition length”: refers to the length over which catheter flexibility changes from flexibility of a first material to flexibility of a second material. Transition length may be measured with various methods: by mechanical properties, optical properties—such as colour—or by chemical composition for instance.

### BRIEF DESCRIPTION OF THE DRAWINGS

FIG. 1 is a schematic of an extruder according to an embodiment: two resin sources are connected to the extrusion die through a commuter element (not shown).

FIG. 2 is a sideview showing an enlargement of extrusion die 11 and commuter element 14, in the first configuration (rotationally sliding). On FIG. 2, thickness of commuter element is not to scale.

### DETAILED DESCRIPTION

The following detailed description will be better understood when read in conjunction with the drawings. For the purpose of illustrating, the device is shown in the preferred embodiments. It should be understood, however that the application is not limited to the precise arrangements, structures, features, embodiments, and aspect shown. The drawings are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

### Numerical References

1: Extruder/2: Barrel/3: Screw drive motor/4: Hopper/5: Feedpipe/11: Die member/12: Mandrel/13: Central cavity/14: Commuter element/15: Inflow channel

This disclosure relates to an extruder 1 conceived to produce hybrid microcatheters. The extruder 1 comprises an extrusion die, comprising a die member 11 and a mandrel 12. Die member 11 and mandrel 12 are disposed along the same direction—namely the extrusion die direction or extrusion die axis. This set-up allows extrusion of a tube comprising a lumen: the mandrel 12 defines the inner dimensions of the tube while the die member 11 defines the outer dimensions of the tube.

In an embodiment, the mandrel 12 has a central cavity 13 that extends in the axial direction and the central cavity 13 of the mandrel 12 is connected to a gas source, configured to flow gas in order to keep the lumen open during extrusion and cooling of microcatheter under extrusion. Alternatively, the extruder 1 may be combined with a cooling tray in order to cool very quickly the microcatheter after extrusion so as to freeze the geometry of the microcatheter and keep the lumen open.

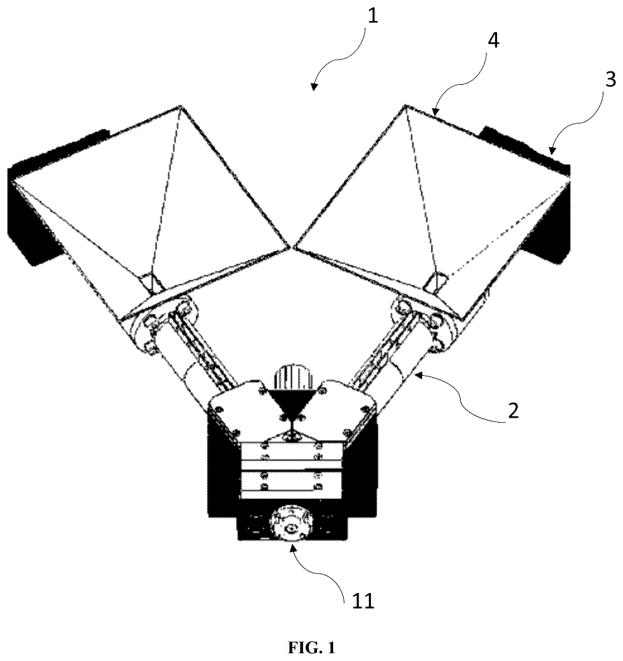

The extruder 1 comprises at least two resin sources. These sources will be used alternately to produce a hybrid microcatheter. As shown on FIG. 1, a resin source typically comprises a hopper 4 in which pellets of a resin—typically a thermoplastic resin or polymer—are introduced; a screw inserted in a heating barrel 2 to melt and move resin towards the extrusion die; and a screw driver 3. FIG. 1 represents two resin sources arranged at an angle of 30° with the axis of extrusion die. In other embodiments, resin sources may be arranged parallel with the axis of extrusion die, or in any suitable direction. More than two sources may be used.

The extruder 1 also comprises a commuter element 14, abutting the extrusion die. The commuter element 14 comprises at least one inflow channel 15 opening on the mandrel 12. During extrusion, resin will be directed from a resin source to the extrusion die through the inflow channel 15.

The commuter element 14 is sliding on the extrusion die. Sliding allows to select resin from one source and to flow said resin through the inflow channel 15 to the mandrel 12, then to the die member 11. The volume delimited by the die member 11, the mandrel 12 and the opening of the inflow channel 15 on the mandrel 11 is an extrusion channel. Extrusion channel prepares resin distribution around the mandrel 12 before the die member 11.

In a first position, the commuter element 14 allows flow of resin from a first resin source. In a second position, the commuter element allows flow of resin from a second resin source. Sliding from first to second position—and vice-versa—allows to change resin fed into the extrusion channel and finally change composition of the microcatheter being extruded.

In this disclosure, the extrusion channel and the inflow channel 15 together have a volume Vᵢₙ lower than 20 mm³. In other words, the sum Vᵢₙ of the volume of extrusion channel and the volume of inflow channel 15 is lower than 20 mm³. Indeed, this volume is related to the amount of resin that will be involved during transition from one resin to another. Extrusion volumes lower than 20 mm³ surprisingly provides with short transition length on hybrid microcatheters, with very good mechanical properties, in particular good resistance to break in the transition.

Preferably, the extruder 1 is conceived to produce thermoplastic hybrid microcatheters, i.e., microcatheters made of thermoplastic polymers.

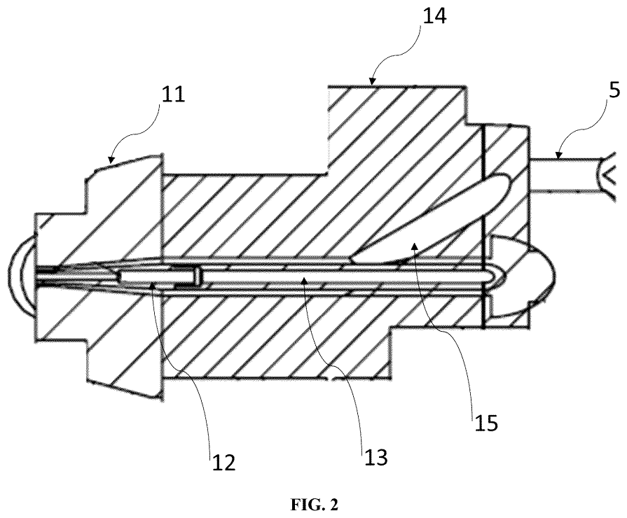

In a first configuration, the commuter element 14 is rotationally sliding on the extrusion die around the extrusion die axis, as shown on FIG. 2.

The inflow channel 15 is here connected to the heating barrel 2 of a resin source via the feedpipe 5. Resin then flows through inflow channel 15, reaches the mandrel 12 and is distributed in the extrusion channel. To select another resin source, commuter element is rotated to align the inflow channel 15 with the feedpipe 5 of said resin source. During sliding, no resin is injected in the inflow channel. In order to avoid extrusion problems, sliding is preferably operated quickly, so that volume of extruded resin remains small as compared to Vᵢₙ during feed interruption duration.

In an embodiment, the extruder 1 comprises three resin sources. Resin sources—referred to as A, B and C—are distributed on a circle, all being connectable with the rotationally sliding commuter element, this embodiment allows to slide from source A to source B; from source A to source C; and from source B to source C. It is thus possible to design any succession of materials along the microcatheter: ABAB; ABCBA; ABCAB, ACBA . . .

In a second configuration, the commuter element 14 is longitudinally sliding on the extrusion die.

For both configurations, the geometry of the microcatheters is defined by the dimensions of the die member 11 on one hand and the dimensions of the mandrel 12 on the other hand. In an embodiment, the diameter of the die member 11 is ranging from 0.4 mm to 2.66 mm—from 1.2 Fr to 8 Fr—and defines the outer diameter of the microcatheter. For instance, the diameter of the die member 11 may be 0.4; 0.5; 0.6; 0.7; 0.8; 0.9; 1; 1.1; 1.2; 1.3; 1.4; 1.5; 1.6; 1.8; 2; 2.2 mm. In an embodiment, the die member 11 has an ellipsoidal or oval shape with dimensions (in mm×mm) 0.8×1.2; 0.6×1; 1×1.4; 1.2×1.6; 1.5×1.9; 1.8×2.2. In an embodiment, the diameter of the mandrel 12 is ranging from 0.15 mm to 2 mm—from about 0.5 Fr to 6 Fr—and defines the inner diameter of the microcatheter, i.e., the lumen diameter. For instance, the diameter of the mandrel may be 0.15; 0.25; 0.4; 0.6; 0.8; 1; 1.4; 1.8 mm. The most suitable geometries for microcatheters are the following couples for die member dimensions/mandrel diameter (in mm): 0.4/0.15; 0.6/0.25; 0.8/0.4; 1/0.6; 1.2/0.8; 1.4/1; 1.6/1.2; 1.8/1.4; 2/1.6 2.2/1.8; 0.8×1.2/0.6; 0.6×1/0.4; 1×1.4/0.8; 1.2×1.6/1; 1.5×1.9/1.2; 1.8×2.2/1.5.

In an embodiment, that may be combined with both configurations, the extrusion die comprises a die member 11 and one single mandrel 12—see FIG. 2. The mandrel 12 may be disposed coaxially with the die member 11, yielding a catheter with a lumen surrounded by walls of uniform thickness. Alternatively, the mandrel 12 may be disposed along the same direction as the die member 11, but off-centered.

In an embodiment, that may be combined with both configurations, the extrusion die comprises a die member 11 and two mandrels 12. The two mandrels 12 are disposed along the same direction as the die member 11. They may be both off-centered. Alternatively, one mandrel 12 is disposed coaxially with the die member and the other mandrel 12 is off-centered. The two mandrels 12 may have the same dimensions or different dimensions. The most suitable geometries for microcatheters are the following triplets for die member dimensions/first mandrel diameter/second mandrel diameter: 0.6/0.15/0.15; 1/0.4/0.4; 1.4/0.6/0.4; 1.8/0.8/0.6; 2.2/1/0.8; 0.8×1.2/0.4/0.4; 1×1.4/0.6/0.4; 1.5×1.9/0.8/0.4.

The invention also relates to a process of extrusion of hybrid microcatheters. This process comprises the following steps.

An extruder 1 as disclosed hereabove is provided and commuter element 14 is placed to allow flow from a first resin source to the die member 11.

Then, a predetermined length of microcatheter is extruded.

In order to change resin source, according to microcatheter design, the commuter element 14 is slidden to allow flow from a second resin source to the die member 11. In order to avoid extrusion problems, sliding is preferably operated quickly, so that volume of extruded resin remains small as compared to Vᵢₙ during feed interruption duration.

Then, another predetermined length of microcatheter is extruded.

In both extrusion steps, extrusion speed may be ranging from 30 mm·s⁻¹ to 200 mm·s⁻¹. In this disclosure, extrusion may be done with gas injection through the mandrel 12, so as to keep the lumen of the microcatheter. Gas flow rate is easily determined by the man skilled in the art. Alternatively, extrusion may be done with a cooling tray intended to freeze microcatheter geometry and keep lumen open during extrusion.

Steps of extrusion and resin source selection may be repeated several times, in order to obtain a catheter with variable properties all along its length.

Preferably, the process of extrusion of hybrid microcatheters is a process using thermoplastic polymers to produce thermoplastic hybrid microcatheters.

The invention also relates to a microcatheter obtained by the process disclosed hereabove. Preferably, the microcatheter is a thermoplastic hybrid microcatheter.

Various microcatheters may be obtained.

Dimensions of microcatheters are defined by geometry of die member 11 and mandrel 12. Outer diameter of microcatheter is ranging from 0.4 mm to 2.66 mm—from 3 Fr to 8 Fr—and preferably the diameter of the die member 11 may be 0.4; 0.5; 0.6; 0.7; 0.8; 0.9; 1; 1.1; 1.2; 1.3; 1.4; 1.5; 1.6; 1.8; 2; 2.2 mm. Inner diameter of microcatheter is ranging from 0.15 mm to 2 mm-from about 0.5 Fr to 6 Fr-and preferably the diameter of the mandrel 12 may be 0.15; 0.25; 0.4; 0.6; 0.8; 1; 1.4; 1.8 mm. The most suitable couples of dimensions for microcatheters are (in mm): 0.4/0.15; 0.6/0.25; 0.8/0.4; 1/0.6; 1.2/0.8; 1.4/1; 1.6/1.2; 1.8/1.4; 2/1.6 2.2/1.8; 0.8×1.2/0.6; 0.6×1/0.4; 1×1.4/0.8; 1.2×1.6/1; 1.5×1.9/1.2; 1.8×2.2/1.5. The most suitable triplets of dimensions for microcatheters are (in mm): 0.4/0.15; 0.6/0.25; 0.8/0.4; 1/0.6; 1.2/0.8; 1.4/1; 1.6/1.2; 1.8/1.4; 2/1.6 2.2/1.8; 0.8×1.2/0.6; 0.6×1/0.4; 1×1.4/0.8; 1.2×1.6/1; 1.5×1.9/1.2; 1.8×2.2/1.5.

Various resin couples, preferably thermoplastic resins, may be used in the process to prepare microcatheters. In each couple, one resin may have a higher flexibility—flexible in the following—whereas the other resin may have a lower flexibility—rigid in the following. For instance, a suitable couple of resins is polypropylene—rigid—and mixture of 50% wt polypropylene with 50% wt of thermoplastic polyurethane—flexible. Mixtures of polypropylene with thermoplastic polyurethane in a weight ratio comprised between 20:80 and 80:20 are suitable as flexible resin for the invention. Another suitable couple of resins is polyamide—rigid—and polyamide-polyether bloc copolymers—flexible—typically a PEBAX® type polymer. Another suitable couple of resins is comprising two grades of polyvinylchloride with different rigidity/flexibility.

In a preferred embodiment, both resins inside a couple of resins have glass transition temperatures having less than 40° C. of difference.

In a preferred embodiment, both resins inside a couple of resins have fusion temperatures having less than 40° C. of difference.

In an embodiment, some resins may be radiopaque. In each resin couple, one resin may be clear, whereas the other resin may comprise markers to be radiopaque.

In an embodiment, three resins, preferably thermoplastic resins, are used: a rigid clear resin, a rigid radiopaque resin and a flexible resin. This embodiment allows to prepare microcatheters with flexibility controlled over its length, the rigid parts of the microcatheters being radiopaque or not.

Alternatively, three resins are used: a rigid resin, a clear flexible resin and a radiopaque flexible resin. This embodiment allows to prepare microcatheters with flexibility controlled over its length, the flexible parts of the microcatheters being radiopaque or not.

As the process allows to change material along the production of a microcatheter with a small volume Vᵢₙ of the extrusion channel and the inflow channel 15, the transition length is usually very short. The selection of Vᵢₙ lower than 20 mm³ leads to transition length shorter than 25 mm usually, preferably lower than 20 mm.

Transition length is the length over which catheter flexibility changes from flexibility of a first material A to flexibility of a second material B. Measurement of transition length may be achieved by various methods.

In a mechanical method, flexibility of the catheter is measured continuously over the length of the catheter and reported in a graph. A transition occurs when flexibility is changing from constant value F_{A} in material A to constant value F_{B} in material B, with a flexibility difference of Δ_{F}. Transition length is here defined as the length over which flexibility varies from 90% of Δ_{F}.

In a chemical method, nature of the material may be measured by spectrometry—typically with InfraRed spectrometry—continuously over the length of the catheter. A transition occurs when spectrum is changing from characteristic spectrum of material A to characteristic spectrum of material B, with a feature different in both spectra. Transition length is here defined as the length over which the amplitude of feature varies from 90%.

For instance, if the couple of resins used is polypropylene and mixture of 50% wt polypropylene and 50% wt of thermoplastic polyurethane: the feature may be the spectral signature of urethane bond in Infra-Red spectroscopy. This feature is absent in polypropylene, whereas it appears at full amplitude in mixture of polypropylene and of thermoplastic polyurethane. For polyamide and polyamide-polyether bloc copolymers, the feature may be the spectral signature of ether bond in Infra-Red spectroscopy.

Transition length may be also evaluated by a colour transition, if material A and material B are tinted with different pigments and or dyes. Transition length is here defined as the length over which the colour varies from colour of material A to colour of material B, with a subjective visual appreciation.

In this disclosure, transition lengths obtained are typically shorter than 30 mm, preferably shorter than 25 mm. The transition length may be even shorter, especially for microcatheters of dimension greater than 1 mm.

### EXAMPLES

The present invention is further illustrated by the following examples.

### Example 1: PP-PP/TPU Microcatheter 1.2 mm/0.8 mm

An extruder with two resin sources and a rotationally commuter element is used. The commuter element is 6 mm thick, with a hole of diameter 1.5 mm in which the mandrel is placed, defining the extrusion channel around the mandrel. Inflow channel is oblique with a diameter of 3 mm. The total volume Vᵢₙ of extrusion channel and the inflow channel together is about 18 mm³.

Resin A is polypropylene (Tg˜−10° C., Tf˜160° C.). Resin B is a mixture of 50% wt of resin A (Tg˜−10° C., Tf˜160° C.) with 50% wt of thermoplastic polyurethane (Tg˜−40° C., Tf>120° C.).

Resin source A is supplying resin with a rotational speed of 45 rpm for the screw, with a temperature of heated barrel at 225° C. Resin source B is supplying resin with a rotational speed of 30 rpm for the screw, with a temperature of heated barrel at 175° C. Temperatures for both resin sources are identical to avoid thermal stresses when resin sources are switched.

Diameter of die member is 1.2 mm and diameter of mandrel is 0.8 mm.

Extrusion starts with resin A, at an extrusion speed of 30 mm·s⁻¹. During extrusion, a constant gas flow rate under pressure of 6 cm H₂O is injected through the mandrel. After few seconds, commuter element is rotated in few ms, in order to switch from resin A to resin B. Extrusion continues for 30 s at an extrusion speed of 30 mm·s⁻¹, then commuter element is rotated back in few ms, in order to select resin A again.

Finally, a 120 cm cm-long microcatheter 1 (4 Fr) is obtained, with two transitions between materials A and B, both transitions having a length shorter than 30 mm.

### Example 2: PP-PP/TPU Microcatheter 1.5 mm/1 mm

Example 1 is reproduced, except a change in extrusion geometry: diameter of die member is 1.5 mm and diameter of mandrel is 1 mm. In addition, resin A is changed for a polypropylene with a glass transition temperature of 175° C., and temperatures for both resin sources are set to 195° C.

Finally, a 120 cm-long microcatheter 2 (5 Fr) is obtained, with two transitions between materials A and B. As diameter and thickness of microcatheter 2 are greater than for microcatheter 1, transitions have shorter lengths, shorter than 25 mm.

### Example 3: A-B-C Microcatheter 1.2 mm/0.8 mm

An extruder with three resin sources and a rotationally commuter element is used. The commuter element is 6 mm thick, with a hole of diameter 1.5 mm in which the mandrel is placed, defining the extrusion channel around the mandrel. Inflow channel is oblique with a diameter of 3 mm. The total volume Vᵢₙ of extrusion channel and the inflow channel together is about 18 mm³.

Resin A is polypropylene (Tg˜−10° C., Tf˜145° C.). Resin B is a mixture of 50% wt of resin A (Tg˜−10° C., Tf˜145° C.) with 50% wt of thermoplastic polyurethane (Tg˜−40° C., Tf>120° C.). Resin C is a polyamid with 20% of BaSO4 radio-opaque marker (Tg˜45° C., Tf˜185° C.).

Temperatures for the three resin sources are identical at 165° C. to avoid thermal stresses when resin sources are switched.

Diameter of die member is 1.2 mm and diameter of mandrel is 0.8 mm.

Extrusion starts with resin A, at an extrusion speed of 30 mm·s⁻¹. During extrusion, a constant gas flow rate under pressure of 6 cm H₂O is injected through the mandrel. After few seconds, commuter element is rotated in few ms, in order to switch from resin A to resin B. Extrusion continues for 15 s at an extrusion speed of 30 mm·s⁻¹, then commuter element is rotated back in few ms, in order to switch from resin B to resin C. Extrusion continues for 15 s, then commuter element is rotated again in few ms, in order to select resin A again.

Finally, a 150 cm-long microcatheter 3 (4 Fr) is obtained, with transitions between materials A and B, then between materials B and C, last between materials C and A. All transitions have a length shorter than 30 mm.

### Example 4: PA-PEBAX Microcatheter 1.2 mm/0.8 mm

Example 1 is reproduced, except a change in resins. Resin A is changed for a polyamide (Tg˜45° C., Tf˜185° C.). Resin B is changed for a polyamide-polyether block copolymer (PEBAX® supplied by Arkema; Tg˜−65° C., Tf˜144° C.), and temperatures for both resin sources are set to 170° C.

Finally, a 165 cm-long microcatheter 4 (4 F Fr) is obtained, with two transitions between materials A and B, both transitions having a length shorter than 30 mm.

### Example 5: PVC-PVC Microcatheter 1.2 mm/0.8 mm

Example 1 is reproduced, except a change in resins. Resin A is changed for a first polyvinylchloride grade (Tg˜78° C.,). Resin B is changed for a second polyvinylchloride, different from the first grade (Tg˜78° C.,), and temperatures for both resin sources are set to 160° C.

Finally, a 165 cm-long microcatheter 5 (4 Fr) is obtained, with two transitions between materials A and B, both transitions having a length shorter than 30 mm.

All microcatheters obtained were tested and show compliant performance results against breakage.

1. An extruder for thermoplastic hybrid microcatheters, the extruder comprising:
an extrusion die comprising a die member and a mandrel, the mandrel and the die member collectively defining an extrusion die axis;
at least two resin sources; and
a commuter element abutting the die member and defining at least one inflow channel having an opening on the mandrel, the commuter element slidable on the extrusion die such that resin from one resin source selected from the at least two resin sources is flowable to the die member through an extrusion channel delimited by the die member, the mandrel and the opening of the inflow channel, the extrusion channel and the inflow channel together have a volume less than 20 mm³, and the mandrel defines a central cavity extending in an axial direction along the extrusion die axis.

2. The extruder of claim 1, wherein the commuter element is rotationally slidable on the extrusion die around the extrusion die axis.

3. The extruder of claim 2, wherein the at least two resin sources comprises three resin sources.

4. The extruder of claim 1, wherein the commuter element is longitudinally slidable on the extrusion die.

5. The extruder of claim 1, wherein dimensions of the die member and of the mandrel are selected in the following couples: 0.4/0.15; 0.6/0.25; 0.8/0.4; 1/0.6; 1.2/0.8; 1.4/1; 1.6/1.2; 1.8/1.4; 2/1.6 2.2/1.8; 0.8×1.2/0.6; 0.6×1/0.4; 1×1.4/0.8; 1.2×1.6/1; 1.5×1.9/1.2; and 1.8×2.2/1.5.

6. A process of extruding a microcatheter, the process comprising:
flowing a first resin from a first source to a die member via a commuter element in a first position abutting the die member;
extruding the first resin along an extrusion axis defined by the die member and a mandrel with the mandrel defining a central cavity in the die member, extrusion of the first resin forming a first predetermined length of the microcatheter;
sliding the commuter element from the first position to a second position abutting the die member;
flowing a second resin from a second source to the die member via the commuter element in the second position; and
extruding the second resin through the die member along the extrusion axis, extrusion of the second resin forming a second predetermined length of the microcatheter.

7. The process of claim 6, wherein the first resin is different from the second resin.

8. The process of claim 7, wherein the first predetermined length and the second predetermined length have different flexibilities.

9. The process of claim 6, wherein extrusion speed of at least one of the first resin or the second resin is between 30 mm/s and 200 mm/s.
